(19) United States
(12) Patent Application Publication
(10) Pub. No.: US 2017/0135609 A1
(43) Pub. Date: May 18, 2017

(54) Relative response system including reprogramming capability for autonomous or interrelated stimulus and sensor systems for measuring biological response data relative to either an absolute reference and/or relative to other biological response

(71) Applicants: Michael Z. Eckblad, Auburn, WA (US); Ken DeMile, Auburn, WA (US)

(72) Inventors: Michael Z. Eckblad, Auburn, WA (US); Ken DeMile, Auburn, WA (US)

(21) Appl. No.: 15/421,368

(22) Filed: Jan. 31, 2017

Related U.S. Application Data
(63) Continuation of application No. 12/460,750, filed on Jul. 24, 2009.
(60) Provisional application No. 61/135,779, filed on Jul. 24, 2008.

(51) Int. Cl. A61B 5/11; A63B 69/32; A61B 5/16

(52) U.S. Cl. CPC A61B 5/1104; A61B 5/162; A63B 69/32

(57) ABSTRACT

A wireless system of a plurality of independent pods initiates, collects, and processes absolute and/or relative response data of a biological system to stimulus. The data is then wirelessly transmitted to any one or combination of external communication devices via a bridge pod. The system is well adapted to collect response measurement data related to a plurality of sequences of stimulus.

### CROSS REFERENCE TO RELATED APPLICATIONS

The present application is a Continuation of co-pending U.S. patent application Ser. No. 12/460,750, filed 2009 Jul. 24, which claims priority under 35 U.S.C. §119(e) from U.S. Prov. Pat. Appl. Ser. No. 61/135,779, filed 2008 Jul. 24, said patent documents being incorporated herein in entirety for all purposes by reference and for which benefit of a chain of priority is hereby claimed.

### GOVERNMENT SUPPORT

Not applicable.

### TECHNICAL FIELD

The present invention relates to systems and methods for collecting biological reaction data to stimuli using microprocessors, sensors, and, more particularly, to using autonomous CPU pods on a wireless mesh network for sensing and processing sequences by first stimulating and then by measuring a biological response.

### BACKGROUND

Measuring a biological reaction to a stimulus is currently problem specific, difficult or impossible to reconfigure, and involves wired data transmission and interconnectivity that interferes with movement. These limitations restrict the ability of coaches, scientists and individuals to understand and optimize the performance of biological systems in a general way, whether it be response of a cow to an electric fence or a response of a boxer to a light or a punch coming his way, or relative reactions between throws and catches on a baseball team, for example.

There are currently in existence some attempts at general study of biological systems. We start with some general examples of products and special application efforts. First is the homemade approach as typified by these quotes from http://www.physicsforums.com/archive/index.php/t-173340.html:

“I was wondering if anyone could help me. I am trying to measure the speed of a karate punch in order to calculate the force of the strike. I have read several papers on the subject but each of these use a high speed video camera and software such a video to analyze the data. Does anyone know of another way to measure the acceleration? I only have a standard camera at 30 frames per second?”

“Measure the total impulse, by striking a heavy bag and seeing how high it swings.”

“If you or one of your friends knows a bit about electronics, it should be quite simple to build a chronograph. Use a couple of strips of aluminum foil a set distance apart as your triggers and punch through them. When you break the first one, the timer starts. The second one stops it.”

The commercial type is illustrated by the “HitMaster” (http://impacttrainingsystems.com/HitMaster.html) which uses various signals and then measures the subject's time to strike a target in response. It will also count the number of hits in a period of time. All data is local to the workout station and all communication is by wired connections. Another type is typified by the Nike or Adidas foot pod which wirelessly sends single user data to a watch or handheld device to be observed by the user.

Finally there are custom research approaches such as seen on “Sports Science” television program, or by large companies or in university studies that rely on custom built sensors, computers, interconnectivity, software, and supporting scientists. These studies are designed to collect data for a specific problem and use it for research or entertainment.

The homemade method suffers from the issues of being non-standardized and having a high variability of accuracy. It avoids any complexities such as wireless or general sensor interface. Thus it is useful only to one end user and his perception of value.

The current commercial versions are centered on a particular application such as boxing or running. Mostly they are wire-connected solutions. Further they tend to be centered on a single subject and workstation and are not scalable to a whole set of stations able to communicate with all the other stations or produce interrelated relative response data among several interrelated subjects. Further, they lack the flexibility to be re-programmed at any time to perform a completely different function and to accommodate any type of sensor and stimulus hardware configurations not previously considered in the initial design.

The research solution in general, despite its possible depth and accuracy, is not usable or affordable by most people or even companies. It is a point solution, custom developed to meet a specific need for data, not a specific solution that is affordable for the average individual or company.

Other patented stimulus-response systems such as described in U.S. Pat. Nos. 6,002,336, 4,941,660, 6,056,674, and 4,534,557 are problem specific and lack flexibility to be reconfigured or measure relative response of interrelated systems needed for commercial success.

### SUMMARY OF THE INVENTION

This invention is unique in that it combines wireless mesh networking technology and independent programming and reprogramming of a plurality of autonomous microprocessor based CPU pods to setup, initiate and measure a sequence of events involving a one to a plurality of biological systems relative to each other and/or relative an absolute reference. Any of the autonomous microprocessor CPU pods can command, initiate, and sense stimulus-response events at any time effectively, making a system whose characteristic function can be changed at any time with simple re-programming. Because of the ability to re-program the CPU pods, the systems provide general accommodation of any sensor or stimulus device. Simple single-event-single-measure systems such as “punch in response to a light” or “counting number of punches in response to a light”, fail to provide the flexibility to measure multiple actions of multiple biological systems against each other and/or relative to an absolute time reference. Further, existing systems do not allow for re-programming the complete character and relative measuring capabilities at any time as part of the normal system function only. Rather, other applications are software or hardware hard-coded from the start to perform one characteristic function. This hard coding of other applications dictates a specific stimulus and sensor hardware configuration as well.

In accordance with the present invention, there is provided a wireless system of autonomous, independent CPU pods for initiating, collecting, and optionally processing, response of one or a plurality of biological systems to stimulus and then using a wireless mesh network and data transfer linkage to transmit or convey the data to a plurality of external communications (such as a notebook or internet address) via a CPU bridge pod and/or to other CPU pods. The system is set up to perform a plurality of re-programmable sequences of stimulus followed by response measurement. The data may be relative to an absolute reference and/or relative to each other. A network of sensors and stimulus devices are connected with the pods according to the purposes of the re-programmable sequences.

It would be advantageous to provide a system of wireless data collection CPU pod(s) that can be re-programmed at any time with behavior over an external communications link such as to a computer.

It would also be advantageous to provide a solution that is scalable from one to a plurality of CPU pods (optionally capable of signal processing) that can have a plurality of stimulus or sensor devices attached and can communicate to each other independently or with all CPU pods simultaneously.

It would also be advantageous to provide a system of independent communicating CPU pods that can be located in close proximity (touching) or miles away while still providing the ability to synchronously or asynchronously collect biological response data to stimuli.

It would be advantageous to use the same CPU pod(s) for initiating a stimulus to trigger a response, optionally for signal processing, and/or for measuring a response, and for then sending the data to the external communications link or to each other.

It would be advantageous to provide a general input and output capability at each CPU pod so that the nodes can be re-programmed at any time to have whatever behavior is desired as a normal operating procedure.

It would further be advantageous to have the relative reaction time of each of a plurality of biological responses referenced to either an absolute clock and/or relative to each other.

### BRIEF DESCRIPTION OF THE DRAWINGS

An understanding of the present invention may be obtained by reference to the accompanying drawings, when considered in conjunction with the subsequent, detailed description, in which:

FIG. 1 is a plan view of a system for gathering time-based stimulus response data of biological systems using multiple autonomous synchronized sensor systems;

FIGS. 2A, 2B, 2C and 2D are a sequence of views illustrating an application of relative reaction sensor to a human and a punching bag acting and reacting while synchronized time and force data is acquired; and,

FIGS. 3A and 3B are detail views of the basic functions of the relative reaction sensor and analysis system in a series of steps of a method.

For purposes of clarity and brevity, like elements and components will bear the same designations and numbering throughout the Figures.

### GLOSSARY

The present invention includes a “relative response system” with reprogramming capability, and with autonomous or interrelated stimulus and sensor systems for measuring biological response data relative to either an absolute reference and/or relative to another biological response. The systems are configured for collecting, and optionally processing, stimulus-response data of subjects (also termed here “biological systems”). The systems may include:

a “wireless network”, for providing data transmission between CPU pod(s) and CPU bridge pod(s) (where the network between the CIP pods is characterized as a mesh network) and also, when needed, a transmission path for software reprogramming of CPU pod(s) and CPU bridge pod(s);

a “CPU pod” with integrated wireless negotiation and microprocessor, power source, independently programmable and reprogrammable instructions in memory or including software, for executing sequences of stimulus-response according to changeable programming—either synchronously or asynchronously and with autonomous or interrelated behavior with other response sensors, other CPU pod(s) or other CPU bridge pod(s) and for communicating on the wireless network;

a “CPU bridge pod”, for linked communication from wireless network to the external communication path as well as perform all functions that a CPU pod performs, and wirelessly connected to the CPU pod;

an “external communication path”, for download and upload of programming and/or data between the CPU bridge pod and a plurality of external communications (e.g., network, computer, human, etc.) electrically connected to the CPU bridge pod for displaying data;

a “stimulus response sequence”, for software- and firmware-coded instructions for executing a plurality independent and/or interrelated stimulus-response actions for an individual biological system or a group of biological systems in order to measure a plurality of biological response data relative to an absolute reference and/or a reference of one response sensor relative to another;

a “trigger device” (also sometimes termed a “stimulus device”), for stimulating any aspect of a biological system including anything a biological system can sense voluntarily or involuntarily, electrically connected to the CPU pod; and,

a “response sensor”, for sensing any aspect of a biological system, electrically connected to the CPU pod. Trigger devices include lights, buzzers, bells, vibrators, shocks, and so forth. Response sensors include load cells, accelerometers, pressure switches, motion sensors, GPS monitors, and so forth.

### DETAILED DESCRIPTION

A wireless system of independent pods is used for initiating, collecting, and optionally processing, absolute and/or relative response data of biological systems to stimulus and then using a wireless mesh network to transmit the data to a plurality of external communication members (e.g. a human interface device) via a bridge pod. The system is set up to perform a plurality of sequences of i) stimulus, followed by ii) response measurement through the wireless network. The elements of the sequence can be changed on each CPU pod via re-programming at any time to create new configurations and metrics. The sequences may execute on each CPU pod(s) and CPU bridge pod(s) autonomously or the programming can create a plurality of different interrelated sequences depending on system objectives. A plurality of sensors and trigger devices are connected to or integrated in the CPU pods according to the purposes of the programmed stimulate response sequences. This system is beneficial for getting stimulus-response data for any biological activity in a relatively independent response manner making it quite general. Thus, interrelated reactions of any number of biological systems relative to each other can be measured, processed and communicated to any desired device for further processing or display. Thus, either individual performance or entire team's relative performance can be tracked to improve performance in a plurality of ways for a plurality of sports, military exercises, and so forth.

### Detailed Description of Components

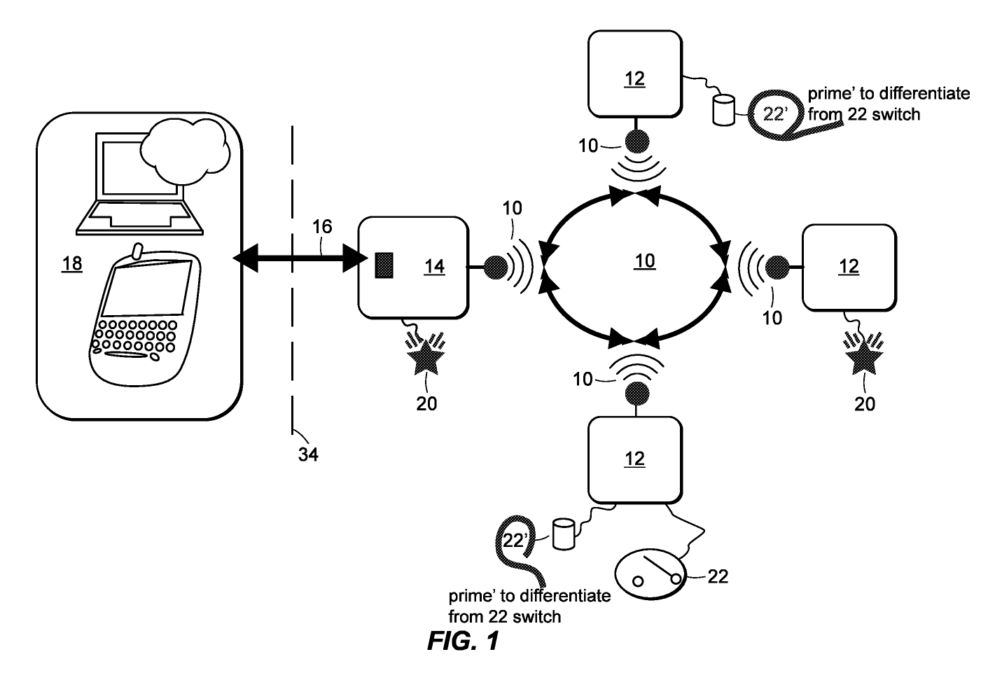

Shown in FIG. 1 are components of a representative system of the invention. The system initiates and collects biological response data either asynchronously or in a synchronized manner according to stimulus response sequence events 28 established within the various CPU pods 12 in the system. The data is then and displayed in a plurality of ways.

The individual components include: wireless network 10: pods 12, bridge pod 14, external communications network link 16, human interface 18 (including smart phones, laptops, internet server, and so forth), trigger device 20 (here suggested as a light; while not limited thereto), and response sensors 22 and 22′ (where 22 is for example an accelerometer and 22′ is a switch having an open and closed state or position).

The system starts with a wireless network 10 of any of a plurality of standards or topologies including all forms of electromagnetic emission or physical signaling including audio, as long as they are capable of transmitting data in or out of CPU pod 12 and CPU bridge pod 14. The wireless network 10 replaces the typical wires used to transmit data in other response measuring systems for biological systems. A preferred wireless network is a mesh network.

The key components of the wireless network 10 are zero or more CPU pods 12 and zero, one or more CPU bridge pod 14(s). The CPU pod 12 may gather data from zero, one or a plurality of sensor devices including switches, accelerometers, heart rate monitor, GPS, and so forth. If there are no sensor devices, the CPU pod 12 may simply act as a repeater for the wireless network 10. The CPU pod 12 has at a minimum a microprocessor, wireless communication hardware and any of a plurality of program elements depending on the desired behavior for the CPU pod 12 and may or may not have resources for data acquisition storage. Optionally, the CPU pod 12(s) can condition a plurality of sensor device signals and then provide the data to the any of the CPU bridge pod 14(s) or to any of the other CPU pod 12(s). Any of the pods may be used to initiate a plurality of synchronous or asynchronous data collection sequences with all or a subset of the other CPU pods or CPU bridge pod 14.

The external communication path 16 provides a means for the CPU bridge pod 14 to communicate outside the wireless network 10 to an external communications 18. A plurality of methods and standards can be used including a wired connection, Bluetooth, or another network such as the Internet or a local area network. For example, the external communications 18 could be co-located with the CPU bridge pod 14 and communicate directly via a Ethernet and a CAT5 cable to a local network or directly to an integrated display or human input device. Another example, the CPU bridge pod 14 resides on the East coast of the USA and a Smart Phone (external communications member 18) on the west coast uses the Internet to connect to the CPU bridge pod 14 via a server integrated into the CPU bridge pod 14 to program the CPU pod 12(s) or CPU bridge pod 14 to have a particular behavior and then uses the same connection to initiate and collect the data for a stimulus response sequence 28.

The external communications 18 is a plurality of devices including PC computer, Internet devices, laptop, PDA (Personal Data Assistant), Smart Phone, etc. that allow a human to input or sense output information from the system. Other forms include non-integrated inputs and outputs such as a separate keyboard and separate LED or LCD display that simply connects directly to the CPU bridge pod 14 to input and/or display output.

The trigger device 20 is used to cause the biological system 24 to respond either voluntarily or involuntarily. The trigger device 20 could stimulate any of the senses or directly drive the nerve system. An example of a stimulate device 20 is a light that turns on to cause a human to throw a punch. Another example is a trigger device that touches a dogs leg (as shown in U.S. Ser. No. 61/135,779, incorporated herein by reference) and initiates a movement response in the dog when turned on. Another example is a neuro-electric device that sends signals directly to a biological systems nerve system.

The response sensor 22 translates biological responses into signal useable by the CPU pod 12. The signals are then used by the CPU pod 12(s) to sense the state of the biological response, for instance, a switch (response sensor 22) that closes (change state) when struck. Another example would be an accelerometer (response sensor 22′) on a human track runner that records acceleration over a period of time (time history). Another example would be a nerve impulse sensor that detects and senses nerve system responses.

### How the Integrated System Works

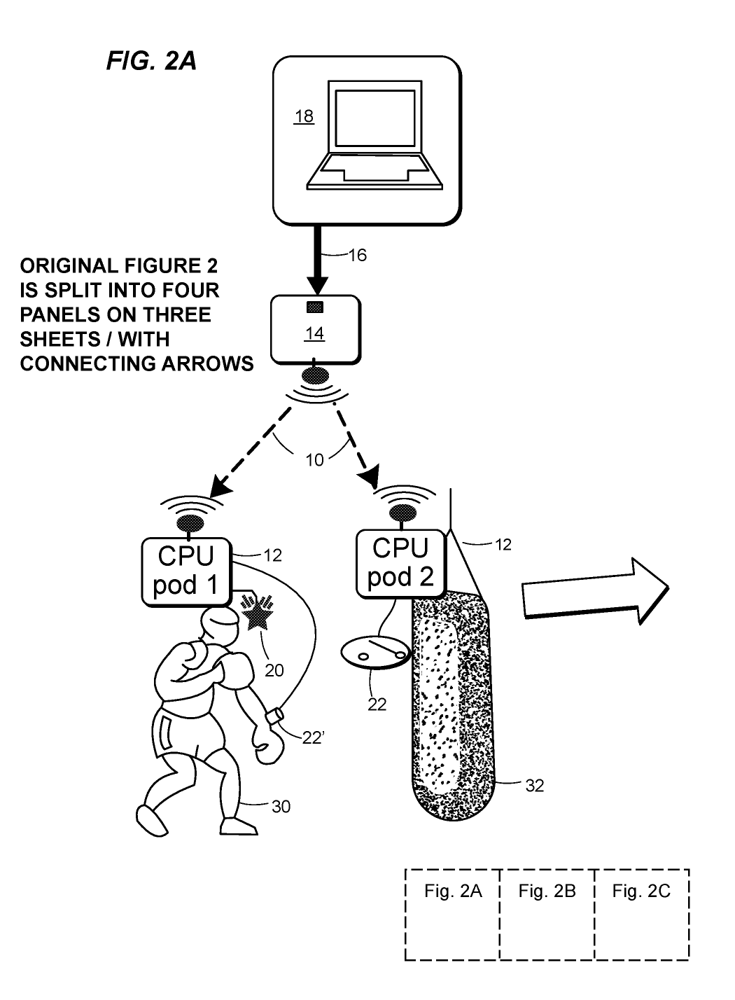

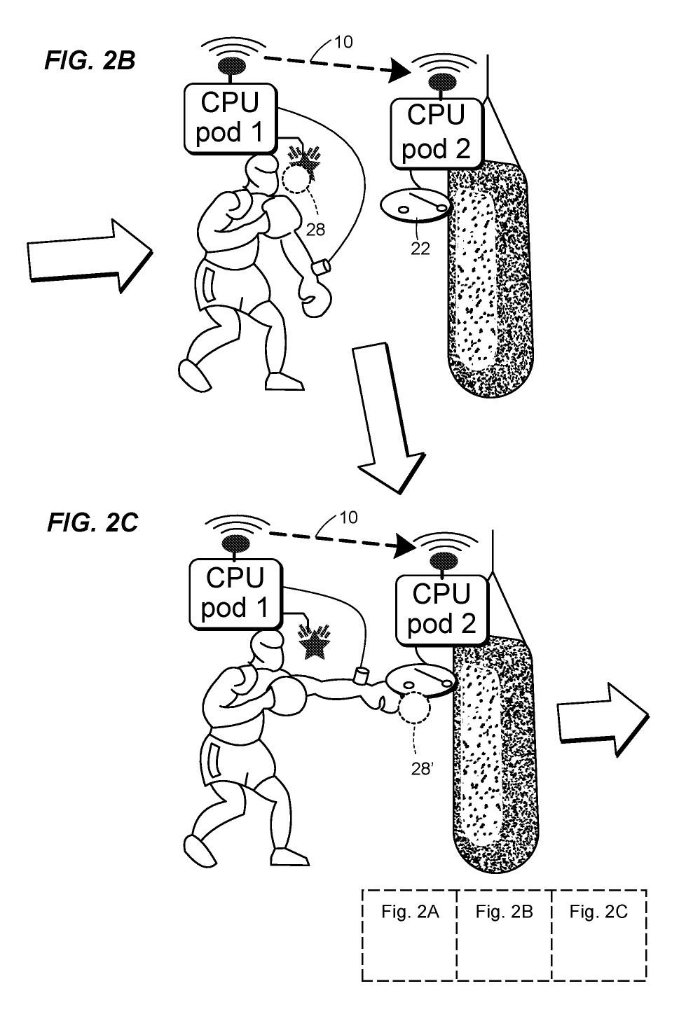

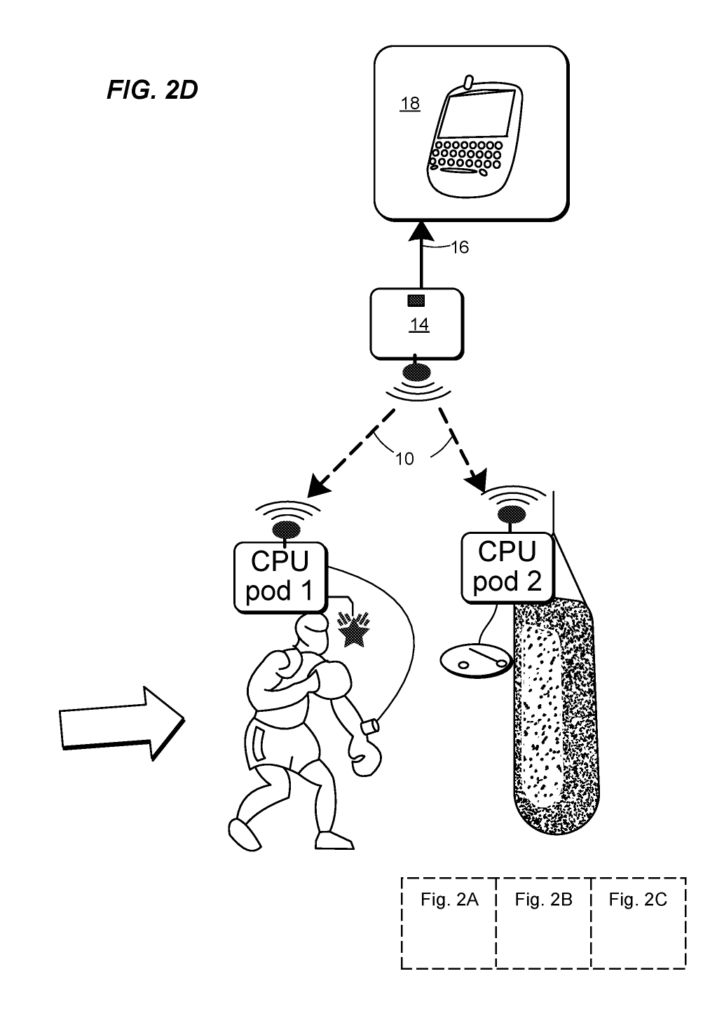

Two examples will be shown here. FIGS. 2A, 2B, 2C and 2D are a sequence of views illustrating an application of a relative reaction sensor in a human interaction with a punching bag. The system and method is set up to measure strike force and synchronized reaction time. The CPU pod 12 and CPU bridge pod 14 drive or read data to and from the attached trigger device(s) 20 and response sensors 22. Typically, the CPU pod may integrate a plurality of sensors directly, including but not limited to GPS location and altitude data, heart rate data, blood pressure, body temperature, and accelerometer. The CPU pod 12 may also include a display or annunciators to communicate with a human and may include a plurality of input or output human interface devices.

The CPU bridge pod 14 may be identical to the CPU pod 12 except it adds an interface for an external communication path 16 to allow data transfer to and from a primary external communications 18. There can be anywhere from 0 to a plurality of CPU bridge pod 14(s) in the system. For example, system can have 0 CPU bridge pod 14(s) when it operates independently of external communications 18. For example, the system could be set up by a human on a notebook (external communications 18) to have one CPU pod 12 turn on a light (stimulate device 20) in 5 minutes and send a signal for another CPU pod 12 to sense 1 minute of velocity data (sensor device) and store it for later use on the CPU pod 12 after the notebook and CPU bridge pod 14 is disconnected. Later, the CPU bridge pod 14 may be reconnected to the wireless network 10 to collect stored data or re-program stimulus response sequence 28. The CPU bridge pod 14 may be involved but is not a requirement for the system to conduct the actual execution of the stimulus response sequence 28. In this example, a CPU pod is attached to an athlete 30 and a punching bag 32 by any of a plurality of methods known in the art.

A programmed stimulus response sequence 28 of events, scheduled to start at time zero, is sent as programming from an external communications device (e.g., a laptop or other human interface device 18) to the CPU bridge pod 14 and then specific sequences are sent individually to CPU pod 1 and CPU pod 2. At time zero, a stimulation device (light 20) is turned on, and CPU pod 1 communicates via the wireless network a start timing command to all involved CPU pod(s). The athlete 30 then hits the punching bag 32 as soon as possible to stop the timer via a switch response device 22 on the punching bag 32 monitored by CPU pod 2. This result is a response time.

At the same time, location and motion of the athlete's hand is tracked by an accelerometer response device 22′ attached to the wrist and fed into CPU pod 1 until CPU pod 2 indicates impact (via wireless network 10). Location data may be monitored until the fist hits the response device switch mounted to the punching bag 32. Part of the data is stored on the CPU pod1 and part of it on CPU pod 2 all synchronized relative to a common time. After the sequence is complete, CPU pod 1 and CPU pod 2 send the data to the CPU bridge pod 14 to be post-processed and then displayed on the external communications 18. As example of reprogramming, a new sequence of events could then be sent over the external communications 18 to cause the light to flash quickly via a new program on CPU pod 1.

At the same time new stimulus response sequence 28 programming is placed on the CPU pod 2 causing it to read time and number of punches per second. After the athlete 30 has completed this sequence, the data from CPU pod 2 (due to the new programming) stores the data on the onboard memory and then at a specified time, sends the data via CPU bridge pod 14 and external communications link 16 over the internet 18 to a specified university internet address for data analysis and display for qualification assessment for the boxer via independent experts at the university.

As a supplemental example of a relative response, a second boxer (not shown) and punching bag with equivalent CPU pod 12(s) could be set up six feet away from the original. The second boxer sequence does not start from time zero but rather relative to and after the first boxer hits his punching bag. Switch response sensor 22 starts timing for the second boxer. This important relative response function illustrates how an entire team's relative performance can be tracked to improve performance in a plurality of ways in a plurality of sports, military performance exercises, and so forth.

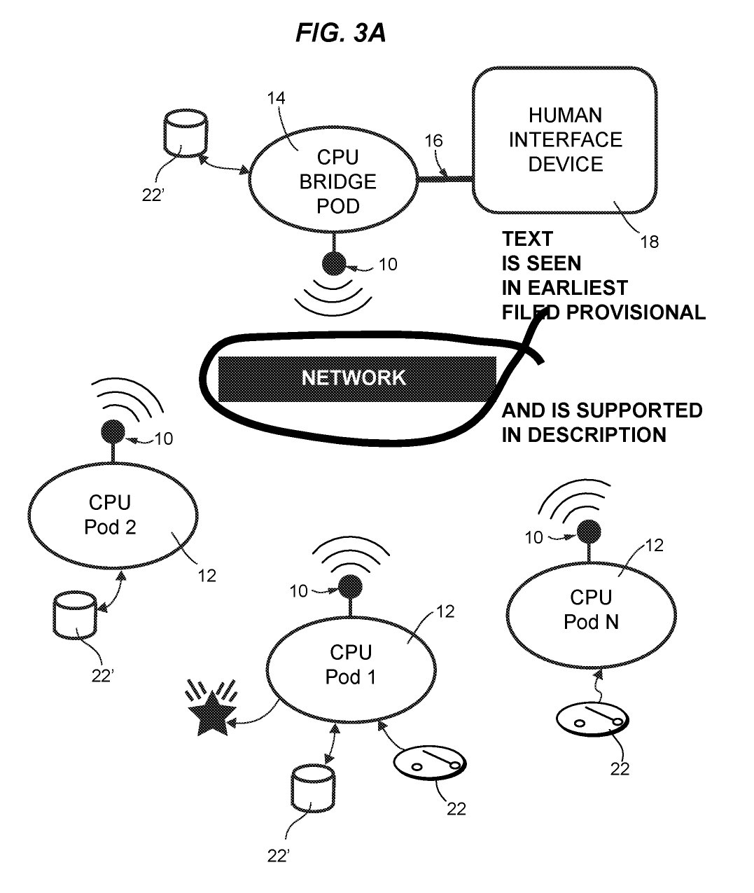

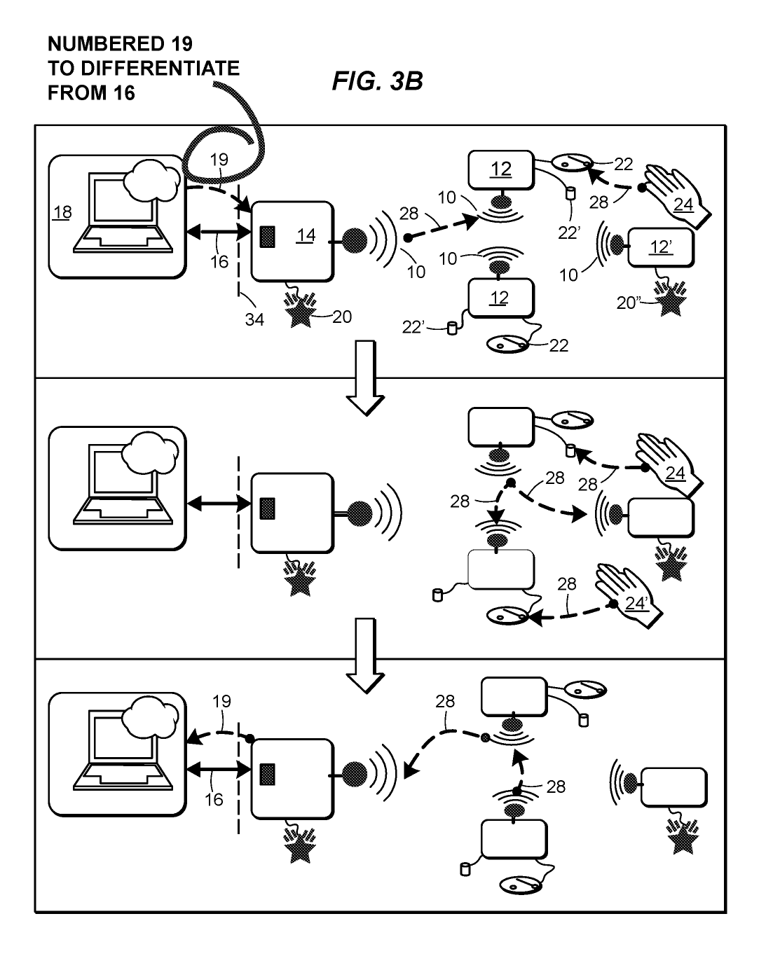

FIGS. 3A and 3B are views of the basic functions of the relative reaction sensor and analysis system in a schematic and as a series of steps of a method. The basic steps of a response sequence are shown.

First, the programming which will support the desired stimulus response sequence events 28 is pushed across the wireless network boundary 34 via external communications path 16 or 19 to a CPU bridge pod 14 from the external communications 18 and from there may be communicated to a plurality of CPU pods forming a mesh network 10.

Second, the sequence is initiated from any of a plurality of conditions such as a specified time, a random time, a particular state of a sensor, a touch, a hit, a throw, a catch, or any signals via the wireless network to all involved CPU pods 12 that the event has started and instructing the pods to initialize the start time.

Third, as shown in FIG. 3B, any of a plurality of trigger device events are executed according to the stimulus response sequence 28 and biological system 24 responses (hand 24, both immediate and consequential) are recorded and optionally signal processed.

The stimulus response sequence 28 may also include dependent sequential initiations of biological systems relative to each other. For instance a pitcher biological system 24 may throw the ball after a light trigger device 20 initiates response while later in the stimulus response sequence 28, the first base biological system 24′ timing starts after he catches the ball for an initiation of response.

Finally, the results are reported to other CPU pods, CPU bridge pods 14, and/or external communications members 18 via links 16 or 19 for any of a plurality of uses including sensory readout (e.g. visual display), further processing, analysis, storage, and/or retransmittal.

Thus, system creates response sensor 22′ input-based sequences via first CPU pod 12 and CPU bridge pod 14 programming that measure the response of a biological system 24 by creating a stimulus 20 and measuring a response as the sequences are executed. This creates a system adaptable to many types of study including athletic, military, animal, and medical applications.

In an example (not shown) having non-locality of the external communications member(s) 18, the system could be set up by a human on a smart phone over the internet (external communications link 19) from one side of town to the wireless network 10 on the other side of town; having one CPU pod 12 turn on a light (trigger device 20) and send a signal for another CPU pod 12′ located on a human to sense heart rate data (sensor device) and send each reading back to the smart phone over the Internet (external communications link 16 or 19) to be plotted out as each point becomes available. At any time the CPU pods in this example can be reprogrammed or the function moved to an entirely different CPU pod 12 or CPU bridge pod 14, allowing a plurality of configurations with the same CPU pod and bridge pod cluster or set.

The integrated system provides a plurality of configurations. Part of the reason for this is that the CPU pods are programmable to perform a plurality of behaviors at any time. This behavior may be completely independent and dependent relative to any other CPU pod. Thus, the methods are not tied to any particular sport or activity or even a specific biological system. These methods seeks to enable a system that can accommodate a plurality of sensors and stimulus devices and can be reconfigured to make any combination of stimulus-response with a simple standard upload of instructions. Because other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

1. (canceled)

2. A relative response system for interrelated stimulus and sensor systems having a plurality of sensors for measuring biological response data based on a time synchronized stimulus, the system comprising:
a wireless network adapted to provide data transmission between at least one CPU pod and at least one CPU bridge pod, the wireless network further adapted to enable a transmission path for software re-programming of any combination of the at least one CPU pod and the at least one CPU bridge pod;
an integrated wireless negotiation and micro-processor coupled to a power source and in data communication with the at least one CPU pod and the at least one CPU bridge pod, the integrated wireless negotiation and micro-processer further configured for executing at least one sequence of a stimulus-response either synchronously or asynchronously;
wherein the at least one CPU bridge pod further comprises a communication connection from the wireless network to an external communication path, and further the at least one CPU bridge pod is adapted to bidirectionally and wirelessly communicate data to the at least one CPU pod;
wherein the external communication path further comprises the ability to upload or download executable programming data between the at least one CPU bridge pod and a plurality of external communication devices;
a stimulate device in data communication with the at least one CPU pod wherein the stimulate device stimulates the biological response;
a response sensor for sensing the biological response, the response sensor in data communication with the at least one CPU pod; and,
wherein the data communication with the at least one CPU pod further comprises time synchronized relative stimulus response data.

3. The system of claim 2 further comprising the at least one CPU pod comprises a plurality of CPU pods wherein each individual CPU pod is enabled with wireless-bidirectional data communication with any combination of the plurality of CPU pods and wherein the wireless, bi-directional data communication further comprises a time-synchronized response data.

4. A wireless training system for improving speed, reaction time, and response metrics of at least one subject's behavior in response to a stimulus, the stimulus and behavior defining a “stimulus-response-sequence event”, which comprises:
a) a plurality of pods, each pod having a microprocessor, a portable power source, a memory with executable program instructions and one or more data caches or buffers, a timer, a counter, supporting circuitry for executing one or more of said program instructions, and a transceiver configured to unite said plurality of pods into a communications network;
wherein at least one of said plurality of pods comprises one of at least one stimulus device, at least one sensor device, or a combination thereof;
wherein said program instructions comprise:
i) an instruction to initiate a stimulus-response-sequence routine at a zero time defined by a start notification, said routine having one or more steps;
ii) an instruction to monitor said sensor device of at least one stimulus device during a stimulus-response-sequence routine, wherein said sensor device is configured to detect a triggered response behavior in response to a trigger stimulus;
iii) an instruction to transmit a stimulus-response-sequence event notification to said network when a triggered response behavior is detected at an elapsed time;
wherein at least one of said plurality of pods is a bridge pod, said bridge pod comprising a transceiver configured to route communication to and from said network and a communications portal configured to push and pull data and one or more stimulus-response-sequence routines from an external path with source; and,
b) a computational module in communication with said network, wherein the computational module is configured to receive, calculate, and report one or more response metrics of a subject's behavior.

5. The training system of claim 4, wherein said program instructions comprise an instruction to synchronize said timer of each of said plurality of pods to a zero time defined by either an absolute clock or a timer in all said pods.

6. The training system of claim 4, wherein said program instructions comprise an instruction to reprogram a stimulus-response-sequence routine when a program notification is received from said network.

7. The training system of claim 6, wherein said program notification comprises an instruction to program a first pod to execute a first step of a stimulus-response-sequence routine and an instruction to program a second pod to execute a second step of a stimulus-response-sequence routine.

8. The training system of claim 4, wherein one pod comprises both a stimulus device and a sensor device; wherein said sensor device is configured to autonomously detect an initial behavior defining a time zero and to initiate a stimulus-response-sequence routine at said time zero; wherein said sensor device is configured to:
a) detect a next response behavior following said time zero;
b) detect a series of response behaviors in said network, each said response behavior following said initial behavior;
c) detect a series of response behaviors in said network, each said response behavior following a subsequent trigger stimulus; or,
d) count a number of response behaviors in an elapsed time.

9. The training system of claim 4, The training system of claim 4, wherein said computational module is housed in at least one said pod and is enabled to perform signal processing and to calculate relative response metrics.

10. The training system of claim 4, wherein said bridge pod comprises a radio connection to at least one pod and a primary digital interface to an external communication link or network.

11. The training system of claim 4, wherein said stimulus device comprises a device for generating a visual stimulus, an audio stimulus, a tactile stimulus or an electrical stimulus to a subject, an athlete, a member of a team, a team, or a pair of contestants.

12. The training system of claim 4, wherein said at least one sensor device is configured to measure and collect sensor data for a metric of a triggered behavior and said instruction to transmit an event notification to said network comprises transmitting at least one metric of said behavior to said network.

13. The wireless training system of claim 4, wherein at least one of said plurality of pods is worn by a subject on a wrist, head or body of a subject.

14. The wireless training system of claim 12, wherein said plurality of pods comprises a first pod configured to generate a trigger stimulus; a second pod configured to detect a response metric, and to transmit a metric to said bridge pod, and to display a response metric to a subject, an athlete, a member of a team, a team, a scientist, a doctor, or a pair of contestants.

15. The training system of claim 12, wherein said computational module is configured to calculate a derived metric from said at least one sensor data, wherein said derived metric is selected from an amplitude, a velocity, a force, a number of behaviors per unit time, a timecourse of a metric, a relative position of a pod over time, a derivative of a motion, an integral of a motion, a heartbeat, a blood pressure, a body temperature, a nerve impulse, a GPS location, or an altitude, and to transmit said derived metric to an external communications link or network.

16. The wireless training system of claim 4, wherein said instruction set comprises an instruction to associate an event notification with a corresponding subject identifier specific for a subject, a team member or a team.

17. The wireless training system of claim 10, wherein said system is configured to display a report of a performance metric or derived metric for a stimulus-response-sequence to a subject, a team member, or a team.

18. The wireless training system of claim 4, wherein said plurality of time-synchronized pods are associated with a single subject and said trigger stimulus is initiated at random, according to a training regimen, or by another subject.

19. The wireless training system of claim 7, wherein said plurality of time-synchronized pods are associated with a team.

20. The wireless training method of claim 18, further comprising a data analysis capability for tracking the behavior of a subject or team over time and detecting an improvement in performance.

21. The wireless training system of claim 4, wherein said plurality of pods comprises a first pod configured to generate a trigger stimulus, a second pod configured to detect accelerometric data in response to said stimulus, and further wherein said system is configured to calculate a result for elapsed time between said stimulus and said response and to display said elapsed time on a display connected to said network.

22. A wireless training method for improving speed, reaction time, and response metrics of at least one subject's behavior in response to a stimulus, the stimulus and behavior defining a “stimulus-response-sequence event”, which comprises:
a) providing a plurality of pods, each pod having a processor, a portable power source, a memory with executable program instructions and one or more data caches or buffers, a timer, a counter, supporting circuitry for executing one or more of said program instructions, a transceiver configured to unite said plurality of pods into a network;
wherein at least one of said plurality of pods comprises at least one stimulus device, at least one sensor device, or a combination thereof;
b) networking said plurality of pods;
c) monitoring said at least one sensor device, wherein said sensor device is configured to detect a triggered behavior in response to a trigger stimulus;
d) upon receiving a start notification, initiating a trigger stimulus, said start notification defining a zero time;
e) transmitting a stimulus-response-sequence event notification to said network when a triggered response behavior is detected at an elapsed time; and,
f) calculating an elapsed time from said start notification to said event notification and report said elapsed time to said network;

23. The wireless training method of claim 22, further comprising at least one bridge pod, said bridge pod comprising a processor, a portable power source, a memory with executable program instructions and one or more data caches or buffers, a timer, a counter, supporting circuitry for executing one or more of said program instructions, a transceiver configured for routing communications to and from said network and a communications portal; and a display.

24. The wireless training method of claim 23, comprising transmitting a behavior response metric with said event notification from said network and displaying said response metric on said bridge pod.

25. The wireless training method of claim 24, comprising associating the event notification with an identifier specific for a subject, a team member or a team.

26. The wireless training system of claim 22, wherein at least one said pod is configured for autonomous operation as part of a mesh network and is configured to initiate a stimulus-response-sequence in response to a behavioral event initiated by a subject.

27. The wireless training system of claim 25, comprising providing a computational module in communication with said network, wherein the computational module is configured to receive, calculate and report one or more behavior response metrics of a subject's behavior.

28. The wireless training method of claim 27, comprising calculating a derived metric from said at least one sensor data for a subject or a team, wherein said derived metric is selected from an amplitude, a velocity, a force, a number of behaviors per unit time, a timecourse of a metric, a relative position of a pod over time, a derivative of a motion, an integral of a motion, a heartbeat, a blood pressure, a body temperature, a nerve impulse, a GPS location, or an altitude, and transmitting said derived metric to an external communications link or network.

29. The wireless training method of claim 28, comprising displaying a report of a performance metric or said derived metric for a stimulus-response-sequence for a subject, a team member, or a team.

30. The wireless method of claim 25, wherein said plurality of pods comprises a first pod configured to generate a trigger stimulus; a second pod configured to detect an accelerometric muscle response to said stimulus, and a third pod configured to detect an accelerometric response of a target, and further wherein said system is configured to derive an elapsed time, solve a transfer function for said accelerometric data into a relative force plot delivered onto said target, and to display said elapsed time and said plot.

31. The wireless training method of claim 22, comprising synchronizing said timers of said plurality of pods to an absolute time or a zero time.

32. The wireless training system of claim 4, wherein at least one said pod is configured for autonomous operation as part of a mesh network and is configured to initiate a stimulus-response-sequence in response to a behavioral event initiated by a subject.

33. The wireless training system of claim 5, wherein said at least one pod having a sensor device is configured to store behavioral response data from said sensor until completion of a stimulus-response-sequence event and to then transmit said data to said bridge pod.

34. The wireless training system of claim 4, wherein at least one of said sensor devices is worn by a subject on a wrist, head or body of a subject and is wiredly or wiredly linked to at least one said microprocessor or said network.

35. The wireless training system of claim 4, wherein at least one of said stimulus devices is worn by a subject on a wrist, head or body of a subject and is wiredly or wiredly linked to at least one said microprocessor or said network.
